(19) United States
(12) Patent Application Publication
(10) Pub. No.: US 2026/0301005 A1
(43) Pub. Date: Oct. 1, 2026

(54) METHOD FOR VALUATION OF UNSTRUCTURED MEDICAL DATA

(71) Applicants: CHA UNIVERSITY INDUSTRY-ACADEMIC COOPERATION FOUNDATION, Pocheon-si (KR); SEOUL NATIONAL UNIVERSITY HOSPITAL, Seoul (KR)

(72) Inventors: Myung-Gwan KIM, Seongnam-si (KR); Hyunwook HAN, Seongnam-si (KR); Hyeong Won YU, Seoul (KR)

(73) Assignees: CHA UNIVERSITY INDUSTRY-ACADEMIC COOPERATION FOUNDATION, Pocheon-si (KR); SEOUL NATIONAL UNIVERSITY HOSPITAL, Seoul (KR)

(21) Appl. No.: 19/474,952

(22) PCT Filed: Apr. 11, 2024
(86) PCT No.: PCT/KR2024/004822

§ 371 Date: Oct. 13, 2025

(30) Foreign Application Priority Data
Apr. 13, 2023 (KR) 10-2023-0048989

(51) Int. Cl. G06Q 30/0201; G16H 50/70

(52) U.S. Cl. CPC G06Q 30/02012; G16H 50/70

(57) ABSTRACT

The present disclosure relates to a method of valuation of unstructured medical data. More specifically, the present disclosure relates to a method for valuing medical data in consideration of its characteristics, in addition to existing methods of evaluating value, such as a market approach, a cost approach, and an income approach. According to a method and server for valuation of the present disclosure, a reasonable value for medical data may be set, thereby preventing unstandardized pricing in medical data exchanges and presenting a reasonable standard. In addition, by establishing reasonable pricing standards, the reasonable pricing standards may be widely used across the rapidly growing medical data market.

### TECHNICAL FIELD

This application claims priority based on Korean Patent Application No. 10-2023-0048989, filed on Apr. 13, 2023, the entire content of which is incorporated herein by reference.

The present disclosure relates to a method of valuation of unstructured medical data.

### BACKGROUND ART

In the era of the Fourth Industrial Revolution, data is considered to be as valuable as crude oil and thus holds a very important position. Among these, medical data may play a key role in the development of medical artificial intelligence (AI) and next-generation medical devices, and efforts to use this medical data are ongoing.

In order to make appropriate use of medical data, attempts being made to trade medical data are increasing, and the number of businesses operating platforms for trading medical data is also increasing. However, since there is no standard for determining a reasonable price for data, it is very difficult to derive an appropriate value of data, and thus consumers are reluctant to trade medical data.

Methods of assessing value of data are being actively researched, and in fact, many systems for valuation of data have been developed. However, unlike general data, due to the specialized nature of medical fields, even when prior art is used, medical data is difficult to value according to general standards. Therefore, as a result of research efforts to develop a method of valuation of medical data, a new method of valuation has been developed that references existing valuation methods while reflecting the specialized characteristics of medical fields.

### DISCLOSURE OF INVENTION

### Technical Problem

An aspect is to provide a method of valuation of medical data, which includes acquiring medical data, valuation of the acquired medical data, and performing a final valuation.

Another aspect is to provide a server for valuation of medical data, which includes a medical data collection unit configured to acquire medical data, a data calculation unit configured to calculate value of the medical data by calculating the value of the medical data acquired according to a cost approach, calculate a scale of an institution in which the medical data is generated, store the medical data, calculate a management cost, calculate the value according to a market approach, and calculate the value according to an income approach, a data provision unit configured to provide a calculated value evaluation result of the medical data to a platform or a mobile device, and a data storage unit configured to store the acquired medical data and the value evaluation result thereof.

However, the problems to be solved by the present disclosure are not limited to the above-described problems, and other problems which are not described herein will be clearly understood from the following description by a person of ordinary skill in the art.

### Solution to Problem

An aspect provides a method of valuation of medical data, which includes acquiring medical data, valuation of the acquired medical data, and performing a final valuation.

In an embodiment, the valuation of the medical data may include operations below:
a) calculating the value according to a cost approach;
d) calculating the value according to a market approach; and
d) calculating the value according to an income approach.

In another embodiment, the calculating the value according to the cost approach may include calculating a scale of an institution in which the medical data is generated, and calculating a weight according to the calculated scale.

In another embodiment, the calculating the value according to the cost approach may further include storing the medical data and calculating a management cost.

In another embodiment, the medical data may be X-ray data.

In another embodiment, the performing of the final valuation may further include classifying in which a disease is identifiable and medical data in which a disease is not identifiable.

In another embodiment, in the final valuation, medical data in which a disease is not identified may be valued as having 60% of value of medical data in which a disease is identified.

In another embodiment, the valuation of the medical data further may include adding a weight to a distribution fee.

In another embodiment, the valuation of the medical data further may include adding value added tax according to data transaction.

In another embodiment, the calculating the value according to the cost approach may include calculating the value according to medical practice unit cost data.

In another embodiment, the valuation according to the market approach may include reflecting a weight according to scarcity of a corresponding disease.

Another aspect provides a server for evaluating the value of medical data, which includes a medical data collection unit configured to acquire medical data, a data calculation unit configured to calculate value of the medical data by calculating the value of the medical data acquired according to a cost approach, calculate a scale of an institution in which the medical data is generated, store the medical data, calculate a management cost, calculate the value according to a market approach, and calculate the value according to an income approach, a data provision unit configured to provide a calculated value evaluation result of the medical data to a platform or a mobile device, and a data storage unit configured to store the acquired medical data and the value evaluation result thereof.

### Advantageous Effects of Invention

According to a method and server for valuation of the present disclosure, a reasonable value for medical data may be set, thereby preventing unstandardized pricing in medical data exchanges and presenting a reasonable standard. In addition, by establishing reasonable pricing standards, the reasonable pricing standards may be widely used across the rapidly growing medical data market.

### BRIEF DESCRIPTION OF DRAWINGS

FIG. 1 is a flowchart illustrating a method of valuation of medical data.

FIG. 2 is a flowchart specifically illustrating X-ray data calculation in the method of evaluating the value of FIG. 1.

FIG. 3 is a block diagram of a system for valuation of medical data, which includes a server for evaluating the value of medical data.

FIG. 4 is a block diagram of a server for valuation of medical data.

### BEST MODE FOR CARRYING OUT THE INVENTION

As used herein, the singular form may include the plural form unless the context clearly indicates otherwise. Further, the terms “comprise and include” and/or “comprising and including” used in this specification should be interpreted as specifying the presence of described shapes, numbers, steps, operations, members, elements, and/or groups thereof and do not exclude the presence or addition of other shapes, numbers, steps, operations, members, elements, and/or groups thereof.

In describing the present disclosure, descriptions of technical contents that are well known in the technical field to which the present disclosure belongs and are not directly related to the present disclosure are omitted. This is to more clearly convey the gist of the present disclosure by omitting unnecessary descriptions without obscuring the gist of the present disclosure. Further, the terms to be described below are terms defined in consideration of functions in the present disclosure and thus may vary according to intentions or customs of users and operators. Accordingly, the definition should be made based on the content throughout the specification.

For the same reason, in the accompanying drawings, some components are exaggerated, omitted, or schematically illustrated. In addition, the size of each component does not completely reflect the actual size thereof. In each drawing, the same reference numerals are used for the same or corresponding components.

The advantages and features of the present disclosure and methods of accomplishing the same will become apparent from the following description of the embodiments in detail, taken in conjunction with the accompanying drawings. However, the present disclosure is not limited to the embodiments to be disclosed below and may be implemented in various different forms. The disclosed embodiments are merely provided so that the present disclosure will be complete and will fully convey the scope of the invention to those skilled in the art. An embodiment of the present disclosure may be defined according to the claims. Like reference numerals refer to like components throughout the specification. In addition, describing an embodiment of the present disclosure, when it is determined that a specific description of a related function or configuration may unnecessarily obscure the gist of the present disclosure, the detailed description thereof will be omitted. Further, the terms to be described below are terms defined in consideration of functions in the present disclosure and thus may vary according to intentions or customs of users and operators. Accordingly, the definition should be made based on the content throughout the specification.

In an embodiment, each block of flowchart drawings and combinations of the flowchart drawings may be performed by computer program instructions. The computer program instructions may be installed on a processor of a general-purpose computer, special-purpose computer, or other programmable data processing apparatus, and the instructions performed through computers or processors of other programmable data processing apparatuses may generate means for performing the functions described in the flowchart block(s). The processor may write data to a memory or read data stored in the memory, and in particular, by executing a program stored in the memory, the processor may process data according to predefined operational rules or an artificial intelligence model. Accordingly, the processor may perform the operations described in the following embodiments, and unless otherwise specified, operations described as being performed by a server in the following embodiments may be regarded as being performed by the processor. The computer program instructions may be stored in a computer usable or readable memory oriented to a computer or other programmable data processing apparatuses, in order to implement functions in a specific method. Thus, the instructions stored in the computer usable or readable memory may be used to manufacture products including instruction units for performing the functions described in the flowchart block(s). The computer program instructions may also be installed on a computer or other programmable data processing apparatuses.

A memory may be a component for storing various programs or data and may include storage media or a combination of storage media such as a read-only memory (ROM), a random-access memory (RAM), a hard disk, a compact disk (CD)-ROM, and a digital video disk (DVD). The memory may not be separately present but may be included in the processor. The memory may include a volatile memory, a nonvolatile memory, or a combination of a volatile memory and a volatile memory. The memory may store a program for performing operations according to embodiments to be described below. The memory may also provide stored data to the processor upon the request of the processor.

In addition, respective blocks of the flowchart may indicate modules, segments, or some of codes including at least one executable instruction for executing a specific logical function(s). In an embodiment, the functions described in the blocks may also occur out of order. For example, two blocks shown in succession may be executed substantially simultaneously, or in reverse order according to functions thereof.

The term “~unit” used in an embodiment of the present disclosure may refer to software or a hardware component such as a field programmable gate array (FPGS) or an application specific integrated circuit (ASIC), and the “~unit” may perform a specific role. Meanwhile, the term “~unit” is not limited to software or hardware. The “~unit” may be formed to be in an addressable storage medium or may be formed to operate one or more processors. In an embodiment, the term “~unit” may include components such as software components, object-oriented software components, class components, and task components, processes, functions, attributes, procedures, subroutines, segments of program code, drivers, firmware, micro-codes, circuits, data, a database, data structures, tables, arrays, and variables. Functions provided by a specific component or a specific “~unit” may be combined to reduce the number of components or may be divided into additional components. In addition, in an embodiment, the “~unit” may include one or more processors.

Hereinafter, embodiments of the present disclosure will be described in detail with reference to the accompanying drawings.

The terms “consist(s) of” or “include(s) (or comprise(s))” should not be interpreted or understood as including, without exception, all of a plurality of components or a plurality of steps disclosed in the specification. In other words, it should be understood that some of the components or some of the steps may not be included, or that additional components or steps may be further included.

Each description should not be construed as limiting the scope of rights, and what can be easily inferred by a person skilled in the art should be construed as belonging to the scope of rights.

Hereinafter, embodiments of the present disclosure will be described in detail with reference to the accompanying drawings.

A method of evaluating the value of medical data of an embodiment relates to a novel method of evaluating value, which is capable of determining value of unstructured medical data in consideration of the special characteristics of medical data based on the method of evaluating value.

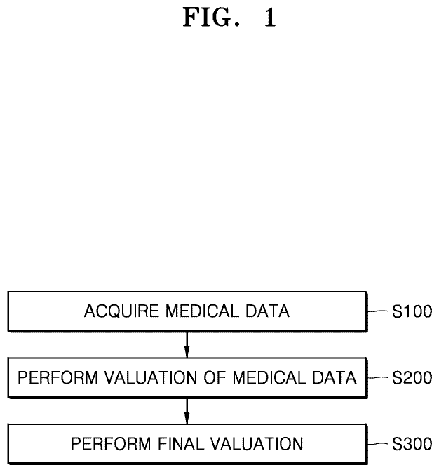

FIG. 1 is a flowchart illustrating a method of valuation of medical data according to an embodiment, wherein S denotes a step.

As shown in FIG. 1, the method of valuation of medical data according to an embodiment includes acquiring medical data (S100), valuation of the medical data (S200), and performing final valuation (S300). In addition, the method of evaluating the value of medical data may be performed by a server 200 for evaluating the value of medical data.

The acquiring of the medical data is not limited to any device as long as the device is a device, a server, or a platform capable of receiving medical data, but preferably, the medical data may be acquired from an X-ray imaging device or a medical data storage device 100 that stores data obtained from X-ray imaging. The medical data is not limited thereto, but may be structured or unstructured data. The unstructured data may include unstructured x-ray data, unstructured voice data, unstructured image data, and unstructured genetic data, and preferably may be unstructured X-ray data.

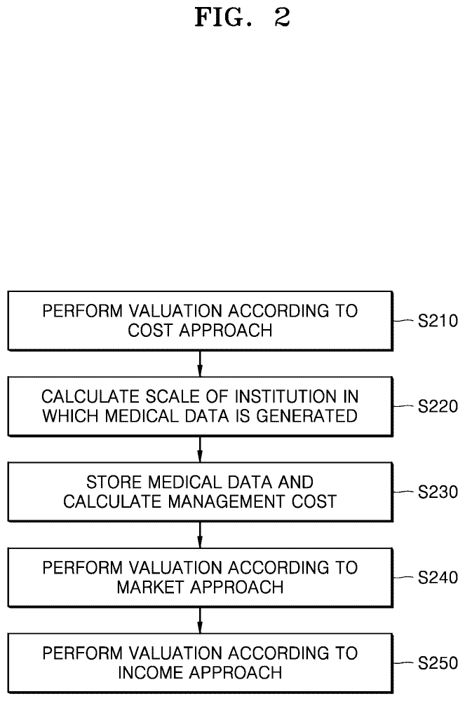

As shown in FIG. 2, the valuation of the medical data (S200) may include operations below:
a) calculating the value according to a cost approach (S210);
b) calculating a scale of an institution in which the medical data is generated (S220);
c) storing the medical data and calculating a management cost (S230);
d) calculating the value according to a market approach (S240); and
e) calculating the value according to an income approach (250).

A specific method of evaluating the value according to an embodiment is shown in Table 1 below.

TABLE 1

| | | | | Unit |
|---|---|---|---|---|
| Calculation | Item | Detailed item | Weight | cost |
| Cost | Practice | Chest iordotic | — | 4,370 |
| approach | | view (1 film) | | |
| | Scale | General | 25% | 5,463 |
| | | hospital_medium | | |
| | | enterprise | | |
| | Consultation | General | — | 17,400 |
| | fee | hospital_medium | | |
| | | enterprise | | |
| | Original data | — | 10% | 24,603 |
| | storage and | | | |
| | management | | | |
| | cost | | | |
| | (scale + | | | |
| | consultation | | | |
| | fee) | | | |
| | Medical | — | — | 24,603 |
| | data cost | | | |
| Market | Quality weight | — | — | 24,603 |
| access | Scarcity weight | Acute bronchitis and | −80% | 4,921 |
| | | acute bronchiolitis | | |
| Income | Effectiveness | 20% added as | 20% | 5,905 |
| approach | weight | expected added | | |
| | | value | | |
| | | obtainable after | | |
| | | technology | | |
| | | development | | |
| | | using medical data | | |
| Reflecting | Weight for | Unstructured X-ray | 10% | 6,495 |
| of | distribution fee | | | |
| distribution | | | | |
| fee | | | | |
| Reflecting | Weigh | Value-added tax | 10% | 7,145 |
| of other | according | | | |
| statutory | to statutory | | | |
| taxes | tax | | | |

Table 1 shows the evaluation of a value of chest X-ray data. In cost approach, a unit cost of a chest iordotic view corresponds to 4,370. The evaluation of the value was initiated based on the unit cost. The unit costs for specific items are shown in Table 2 below. Table 2 below was made based on data from the KOICD Disease Classification Information Center. However, the following data may be appropriately modified and applied based on foreign disease classification data.

TABLE 2

| Practice | Classification | Unit cost |
|---|---|---|
| Chest lordotic view (1 film) | Simple radiological | 4,370 |
| | diagnostic fee | |
| Abdomen (1 film) | Simple radiological | 4,170 |
| | diagnostic fee | |
| Chest [direct] (1 film) | Simple radiological | 6,390 |
| | diagnostic fee | |
| Head (1 film) | Simple radiological | 4,100 |
| | diagnostic fee | |
| Fibula (1 film) | Simple radiological | 4,090 |
| | diagnostic fee | |
| Orbit [double exposure] | Simple radiological | 4,170 |
| (1 film) | diagnostic fee | |
| Paranasal sinus (1 film) | Simple radiological | 4,150 |
| | diagnostic fee | |
| Mastoid and ossicle | Simple radiological | 4,150 |
| (1 film) | diagnostic fee | |
| Mandible (1 film) | Simple radiological | 4,090 |
| | diagnostic fee | |
| Optic foramen (1 film) | Simple radiological | 4,090 |
| | diagnostic fee | |
| Temporomandibular joint | Simple radiological | 4,170 |
| (1 film) | diagnostic fee | |
| Sella (1 film) | Simple radiological | 4,090 |
| | diagnostic fee | |
| Neck, soft tissue (1 film) | Simple radiological | 4,090 |
| | diagnostic fee | |
| Maxilla (1 film) | Simple radiological | 4,170 |
| | diagnostic fee | |
| Zygomatic arch view | Simple radiological | 4,100 |
| (1 film) | diagnostic fee | |
| Plain radiograph of | Simple radiological | 4,170 |
| the kidneys, ureters, | diagnostic fee | |
| and bladder (1 film) | | |
| Breast-mammography (1 film) | Simple radiological | 1,2300 |
| | diagnostic fee | |
| Breast-magnification | Simple radiological | 2,1750 |
| mammography (1 film) | diagnostic fee | |
| Clavicle (1 film) | Simple radiological | 5,090 |
| | diagnostic fee | |
| Scapula (1 film) | Simple radiological | 5,090 |
| | diagnostic fee | |
| Shoulder joint (1 film) | Simple radiological | 5,090 |
| | diagnostic fee | |
| Sternum (1 film) | Simple radiological | 5,170 |
| | diagnostic fee | |
| Rib (1 film) | Simple radiological | 5,180 |
| | diagnostic fee | |
| Acromioclavicular joint | Simple radiological | 5,090 |
| (1 film) | diagnostic fee | |
| Sternoclavicular joint | Simple radiological | 5,090 |
| (1 film) | diagnostic fee | |
| Cervical spine (1 film) | Simple radiological | 5,180 |
| | diagnostic fee | |
| Cervicothoracic spine | Simple radiological | 5,180 |
| (1 film) | diagnostic fee | |
| Thoracic spine (1 film) | Simple radiological | 5,180 |
| | diagnostic fee | |
| Thoracolumbar spine (1 film) | Simple radiological | 5,180 |
| | diagnostic fee | |
| Lumbar spine (1 film) | Simple radiological | 5,180 |
| | diagnostic fee | |
| Lumbosacral spine (1 film) | Simple radiological | 5,180 |
| | diagnostic fee | |
| Sacral spine and coccyx | Simple radiological | 5,180 |
| (1 film) | diagnostic fee | |
| Entire spine (1 film) | Simple radiological | 5,250 |
| | diagnostic fee | |
| Sacroiliac joint (1 film) | Simple radiological | 5,090 |
| | diagnostic fee | |
| Pelvis (1 film) | Simple radiological | 5,180 |
| | diagnostic fee | |
| Hip joint (1 film) | Simple radiological | 5,090 |
| | diagnostic fee | |
| Infantogram (1 film) | Simple radiological | 10,760 |
| | diagnostic fee | |
| Long bone (1 film) | Simple radiological | 4,530 |
| | diagnostic fee | |
| Forearm (1 film) | Simple radiological | 4,530 |
| | diagnostic fee | |
| Elbow (1 film) | Simple radiological | 4,530 |
| | diagnostic fee | |
| Humerus (1 film) | Simple radiological | 4,530 |
| | diagnostic fee | |
| Wrist (1 film) | Simple radiological | 4,530 |
| | diagnostic fee | |
| Hand (1 film) | Simple radiological | 4,530 |
| | diagnostic fee | |
| Carpal bone (1 film) | Simple radiological | 4,530 |
| | diagnostic fee | |
| Femur (1 film) | Simple radiological | 4,530 |
| | diagnostic fee | |
| Knee joint (1 film) | Simple radiological | 4,530 |
| | diagnostic fee | |
| Lower leg (1 film) | Simple radiological | 4,530 |
| | diagnostic fee | |
| Ankle (1 film) | Simple radiological | 4,530 |
| | diagnostic fee | |
| Foot (1 film) | Simple radiological | 4,530 |
| | diagnostic fee | |
| Scanogram (1 film) | Simple radiological | 4,870 |
| | diagnostic fee | |
| Patella (1 film) | Simple radiological | 4,530 |
| | diagnostic fee | |
| Tarsal bone (1 film) | Simple radiological | 4,530 |
| | diagnostic fee | |
| 7Calcaneus (1 film) | Simple radiological | 4,530 |
| | diagnostic fee | |
| Finger (1 film) | Simple radiological | 4,530 |
| | diagnostic fee | |
| Toe (1 film) | Simple radiological | 4,530 |
| | diagnostic fee | |
| Periapical view (1 film) | Simple radiological | 3,380 |
| | diagnostic fee | |
| Bitewing view (1 film) | Simple radiological | 3,980 |
| | diagnostic fee | |
| Occlusal view (1 film) | Simple radiological | 4,040 |
| | diagnostic fee | |
| Conventional panoramic view | Simple radiological | 1,0870 |
| | diagnostic fee | |
| Special panoramic view | Simple radiological | 12,300 |
| (temporomandibular | diagnostic fee | |
| joint/mandibular section) | | |
| Cephalometric view | Simple radiological | 8,430 |
| | diagnostic fee | |
| Transcranial view | Simple radiological | 16,080 |
| | diagnostic fee | |
| Upper gastrointestinal | Special radiological | 35,610 |
| series | imaging fee | |
| Esophagography | Special radiological | 27,070 |
| | imaging fee | |
| Female genitalia- | Special radiological | 66,090 |
| hysterosalpingography | imaging fee | |

In the calculating of the value according to the cost approach, the scale of the institution in which the medical data is generated may be confirmed, and then, a weight corresponding to the confirmed scale may be assigned. In the case of the method of evaluating the value according to an embodiment, since value was valued on medical data generated by a general hospital_medium enterprise, and a weight of 25% was calculated. Specific standards are shown in Table 3 below. The weight corresponds to a weight based on a differential inpatient nursing management fee system.

TABLE 3

| Scale | Weight |
|---|---|
| Medical clinic-small enterprise | 15% |
| Hospital_small enterprise | 20% |
| General hospital_medium enterprise | 25% |
| Tertiary general hospital_large enterprise | 30% |

Meanwhile, a consultation fee was calculated based on the KOICD fee code, and in the case of an embodiment corresponding to a general hospital-medium enterprise, the consultation fee was calculated as 17,400. Specific standards thereof are shown in Table 4 below.

TABLE 4

| Scale | Consultation fee_evaluation fee |
|---|---|
| Medical clinic-small enterprise | 15,690 |
| Hospital_small enterprise | 15,640 |
| General hospital_medium enterprise | 17,400 |
| Tertiary general hospital_large | 19,160 |
| enterprise | |

In the calculating of the value according to the market approach (S220), a quality weight and a scarcity weight were calculated However, a specific weight was not selected as the quality weight in value calculation, and only the scarcity weight was calculated. A negative weigh was applied by classifying the scarcity weigh into G1 to G7 according to a proportion of data with the same value, and standards were classified according to the number of treated patients based on the 2018 disease classification. Specific classification standards are shown in Tables 5 and 6 below.

TABLE 5

| Classification | Scarcity weight | Distribution | Notes |
|---|---|---|---|
| G1 | −80% | >=6.00% | Very high |
| G2 | −70% | 3.00-5.00% | High |
| G3 | −60% | 1.00-2.00% | Relatively high |
| G4 | −50% | 0.70-0.99% | Average |
| G5 | −40% | 0.40-0.69% | Relatively low |
| G6 | −30% | 0.10-0.39% | Low |
| G7 | −20% | <=0.09 | Very low |

TABLE 6

| | | Number of | | |
|---|---|---|---|---|
| | | treated | | |
| | | patients | | |
| | | (inpatients, | | Scarcity |
| Disease | Practice | outpatients) | Classification | weight |
| Other disorders of | Conventional | 20,232,388 | G1 | −80% |
| teeth and supporting | panoramic view | | | |
| structures | | | | |
| Acute bronchitis and | Chest [direct] | 17,208,165 | G1 | −80% |
| acute bronchiolitis | (1 film) | | | |
| Other acute upper | Chest iordotic view | 12,200,027 | G2 | −70% |
| respiratory | (1 film) | | | |
| infections | | | | |
| Other diseases | | 12,160,858 | G2 | −70% |
| of the skin and | | | | |
| subcutaneous tissue | | | | |
| Acute pharyngitis | Chest iordotic view | 9,454,074 | G2 | −70% |
| and acute | (1 film) | | | |
| tonsillitis | | | | |
| Other diseases of | Paranasal sinus | 8,439,661 | G2 | −70% |
| nose and nasal | (1 film) | | | |
| sinuses | | | | |
| Soft tissue disorders | Neck, soft tissue | 8,207,956 | G2 | −70% |
| | (1 film) | | | |
| Dislocations, sprains | Elbow (1 film) | 7,820,773 | G3 | −60% |
| and strains of | | | | |
| specified and | | | | |
| multiple | | | | |
| body regions | | | | |
| Other dorsopathies | Entire spine (1 film) | 7,418,938 | G3 | −60% |
| Other symptoms, | | 6,950,570 | G3 | −60% |
| signs, and abnormal | | | | |
| clinical and laboratory | | | | |
| findings, not | | | | |
| elsewhere classified | | | | |
| Essential (primary) | | 6,063,677 | G3 | −60% |
| hyper-tension | | | | |
| Dental caries | Conventional | 5,871,862 | G3 | −60% |
| | panoramic view | | | |
| Gastritis and | Upper gastrointestinal | 5,336,838 | G3 | −60% |
| duodenitis | series | | | |
| Other diseases of | Abdomen (1 film) | 5,276,669 | G3 | −60% |
| intestines and | | | | |
| peritoneum | | | | |
| Conjunctivitis and | | 5,187,923 | G3 | −60% |
| other disorders of | | | | |
| conjunctiva | | | | |
| Other diseases of | Esophagography | 5,159,893 | G3 | −60% |
| oesophagus, stomach | | | | |
| and duodenum | | | | |
| Other injuries of | | 4,847,258 | G3 | −60% |
| specified, unspecified | | | | |
| and multiple body | | | | |
| regions | | | | |
| Othergastroenteritis | | 4,625,172 | G3 | −60% |
| and colitis | | | | |
| of infectious | | | | |
| and unspecified origin | | | | |
| Other diseases of the | | 4,385,600 | G3 | −60% |
| eye and adnexa | | | | |
| Arthrosis | Knee joint (1 film) | 3,870,424 | G3 | −60% |
| Infections of the skin | | 3,746,120 | G3 | −60% |
| and subcutaneous | | | | |
| Bronchitis, | Chest iordotic view (1 | 3,547,582 | G3 | −60% |
| emphysema and other | film) | | | |
| chronic obstructive | | | | |
| pulmonary diseases | | | | |
| Acute laryngitis and | Chest iordotic view (1 | 3,440,222 | G3 | −60% |
| tracheitis | film) | | | |
| Mycoses | | 3,374,902 | G3 | −60% |
| Other diseases of the | Mastoid and ossicle (1 | 3,301,435 | G3 | −60% |
| ear and mastoid | film) | | | |
| process | | | | |
| Diabetes mellitus | | 3,042,943 | G3 | −60% |
| Cervical and other | Lumbar spine (1 film) | 2,768,995 | G3 | −60% |
| intervertebral disc | | | | |
| disorders | | | | |
| Influenza | Chest [direct] (1 film) | 2,714,520 | G3 | −60% |
| Disorders of refraction | | 2,594,785 | G4 | −50% |
| and accommodation | | | | |
| Other endocrine, | | 2,507,387 | G4 | −50% |
| nutritional and | | | | |
| metabolic disorders | | | | |
| Otitis media and other | Mastoid and ossicle (1 | 2,420,386 | G4 | −50% |
| disorders of middle | film) | | | |
| ear and mastoid | | | | |
| Inflammation of eyelid | | 2,273,361 | G4 | −50% |
| (Keratitis and other | | 2,266,352 | G4 | −50% |
| disorder of sclera and | | | | |
| cornea | | | | |
| Chronic sinusitis | Paranasal sinus (1 | 2,175,722 | G4 | −50% |
| | film) | | | |
| Other diseases of | Chest iordotic view (1 | 2,139,304 | G4 | −50% |
| upper respiratory tract | film) | | | |
| Other disorders of | Hip joint (1 film) | 2,132,419 | G4 | −50% |
| joints | | | | |
| Other inflammatory | Pelvis (1 film) | 1,804,827 | G5 | −40% |
| diseases of female | | | | |
| pelvic organs | | | | |
| Rheumatoid arthritis | Shoulder joint (1 film) | 1,762,406 | G5 | −40% |
| and other | | | | |
| inflammatory | | | | |
| polyarthropathies | | | | |
| Abdominal and pelvic | Pelvis (1 film) | 1,739,052 | G5 | −40% |
| pain | | | | |
| Cystitis | Plain radiograph of the | 1,638,623 | G5 | −40% |
| | kidneys, ureters, and | | | |
| | bladder (1 film) | | | |
| Other viral diseases | | 1,506,763 | G5 | −40% |
| Other diseases of the | Plain radiograph of the | 1,476,404 | G5 | −40% |
| urinary system | kidneys, ureters, and | | | |
| | bladder (1 film) | | | |
| Gastric and duodenal | Upper gastrointestinal | 1,473,126 | G5 | −40% |
| ulcer | series | | | |
| Asthma | Chest [direct] (1 film) | 1,438,089 | G5 | −40% |
| Other in situ and | | 1,393,701 | G5 | −40% |
| benign neoplasms and | | | | |
| neoplasms of | | | | |
| uncertain and | | | | |
| unknown behaviour | | | | |
| Cataract and other | | 1,355,785 | G5 | −40% |
| disorders of lens | | | | |
| Persons encountering | | 1,355,561 | G5 | −40% |
| health services for | | | | |
| specific procedures | | | | |
| and health care | | | | |
| Pneumonia | Chest [direct] (1 film) | 1,339,936 | G5 | −40% |
| Other diseases of liver | Chest [direct] (1 film) | 1,276,777 | G5 | −40% |
| Hyperplasia of | Plain radiograph of the | 1,275,069 | G5 | −40% |
| prostate | kidneys, ureters, and | | | |
| | bladder (1 film) | | | |
| Other diseases of the | Mandible (1 film) | 1,271,737 | G5 | −40% |
| oral cavity, salivary | | | | |
| glands and jaws | | | | |
| Other disorders of | Plain radiograph of the | 1,209,413 | G5 | −40% |
| genitourinary tract | kidneys, ureters, and | | | |
| | bladder (1 film) | | | |
| Inflammatory disease | Female genitalia- | 1,205,783 | G5 | −40% |
| of cervix uteri | hysterosalpingography | | | |
| Other intestinal | | 1,160,240 | G5 | −40% |
| infectious diseases | | | | |
| Other disorders of | | 1,153,529 | G5 | −40% |
| thyroid | | | | |
| Other diseases of the | | 1,150,982 | G5 | −40% |
| nervous system | | | | |
| Fractures of other limb | Lower leg (1 film) | 1,115,580 | G5 | −40% |
| Migraine and other | | 1,084,961 | G5 | −40% |
| head-ache syndromes | | | | |
| Other diseases of the | Chest [direct] (1 film) | 1,078,368 | G5 | −40% |
| respiratory system | | | | |
| Disorders of bone | Femur (1 film) | 1,033,824 | G6 | −30% |
| density and structure | | | | |
| Neurotic, stress- | | 1,028,196 | G6 | −30% |
| related and | | | | |
| somatoform disorders | | | | |
| Glaucoma | | 909,350 | G6 | −30% |
| Herpesviral infections | | 900,274 | G6 | −30% |
| Persons encountering | | 899,910 | G6 | −30% |
| health services for | | | | |
| other reasons | | | | |
| Mood [affective] | | 892,436 | G6 | −30% |
| disorders | | | | |
| Disorders of breast | Breast-mammography | 863,692 | G6 | −30% |
| | (1 film) | | | |
| Effects of foreign body | Abdomen (1 film) | 862,369 | G6 | −30% |
| entering through | | | | |
| natural orifice | | | | |
| Other ischaemic heart | | 827,394 | G6 | −30% |
| diseases | | | | |
| Varicella and zoster | | 812,058 | G6 | −30% |
| Other persons with | | 792,201 | G6 | −30% |
| potential health | | | | |
| hazards related to | | | | |
| communicable | | | | |
| disease | | | | |
| Nerve, nerve root and | | 777,046 | G6 | −30% |
| plexus disorders | | | | |
| Fever of unknown | | 761,448 | G6 | −30% |
| origin | | | | |
| Other mental and | | 760,333 | G6 | −30% |
| behavioural disorders | | | | |
| Fracture of skull and | Cephalometric view | 702,739 | G6 | −30% |
| facialbones | | | | |
| Menopausal and other | | 688,910 | G6 | −30% |
| perimenopausal | | | | |
| disorders | | | | |
| Hemorrhoids | | 633,970 | G6 | −30% |
| Disorders of | | 606,207 | G6 | −30% |
| menstruation | | | | |
| Burns and corrosion | | 602,977 | G6 | −30% |
| Persons encountering | | 593,525 | G6 | −30% |
| health services for | | | | |
| examination and | | | | |
| investigation | | | | |
| Antenatal screening | | 585,671 | G6 | −30% |
| and other supervision | | | | |
| of pregnancy | | | | |
| Hearing loss | | 583,164 | G6 | −30% |
| Fracture of neck, | Cervicothoracic spine | 518,727 | G6 | −30% |
| thorax or pelvis | (1 film) | | | |
| Dementia | | 513,746 | G6 | −30% |
| Chronic disease of | | 513,147 | G6 | −30% |
| tonsils and adenoids | | | | |
| Other infections with a | | 502,907 | G6 | −30% |
| predominantly sexual | | | | |
| mode of transmission | | | | |
| Cerebral infarction | Head (1 film) | 484,098 | G6 | −30% |
| Other viral hepatitis | | 482,339 | G6 | −30% |
| Certain early | | 445,631 | G6 | −30% |
| complications of | | | | |
| trauma and | | | | |
| complications of | | | | |
| surgical and medical | | | | |
| care, not elsewhere | | | | |
| classified | | | | |
| Other cerebrovascular | Head (1 film) | 425,333 | G6 | −30% |
| diseases | | | | |
| Conduction disorders | Chest [direct] (1 film) | 412,327 | G6 | −30% |
| and cardiac | | | | |
| arrhythmias | | | | |
| Malignant neoplasm of | | 394,111 | G6 | −30% |
| other, ill-defined, | | | | |
| secondary, | | | | |
| unspecified and | | | | |
| multiple sites | | | | |
| Leiomyoma of uterus | Female genitalia- | 392,894 | G6 | −30% |
| | hysterosalpingography | | | |
| Benign neoplasms of | | 365,028 | G6 | −30% |
| skin | | | | |
| Other hypertensive | | 359,822 | G6 | −30% |
| diseases | | | | |
| Iron deficiency anemia | | 350,654 | G6 | −30% |
| Other diseases of the | | 313,382 | G6 | −30% |
| musculoskeletal | | | | |
| system and | | | | |
| connective tissue | | | | |
| Other diseases of the | | 305,960 | G6 | −30% |
| digestive system | | | | |
| Benign neoplasm of | Breast-mammography | 294,250 | G6 | −30% |
| breast | (1 film) | | | |
| Urolithiasis | Abdomen (1 film) | 292,552 | G6 | −30% |
| Other diseases of | | 288,870 | G6 | −30% |
| male genital organs | | | | |
| Liveborn infants | | 287,061 | G6 | −30% |
| according to place of | | | | |
| birth | | | | |
| Other disorders of | | 286,950 | G6 | −30% |
| prostate | | | | |
| Renal tubulo- | Abdomen (1 film) | 275,919 | G6 | −30% |
| interstitial diseases | | | | |
| Renal failure | Plain radiograph of the | 267,991 | G6 | −30% |
| | kidneys, ureters, and | | | |
| | bladder (1 film) | | | |
| Other complications of | | 254,294 | G7 | −20% |
| pregnancy and | | | | |
| delivery | | | | |
| Other diseases of the | Abdomen (1 film) | 251,975 | G7 | −20% |
| circulatory system | | | | |
| Thyrotoxicosis | | 250,812 | G7 | −20% |
| Intracranial injury | Head (1 film) | 243,208 | G7 | −20% |
| Other peripheral | | 235,364 | G7 | −20% |
| vascular diseases | | | | |
| Other and unspecified | | 225,201 | G7 | −20% |
| effects of external | | | | |
| causes | | | | |
| Cholelithiasis and | Abdomen (1 film) | 225,074 | G7 | −20% |
| cholecystitis | | | | |
| Postpartum care and | | 192,922 | G7 | −20% |
| examination | | | | |
| Other bacterial | | 189,993 | G7 | −20% |
| diseases | | | | |
| Varicose veins of | | 185,676 | G7 | −20% |
| lower extremities | | | | |
| Malignant neoplasm of | Breast-magnification | 182,096 | G7 | −20% |
| breast | mammography (1 film) | | | |
| Other anaemias | | 178,886 | G7 | −20% |
| Strabismus | | 166,263 | G7 | −20% |
| Malignant neoplasm of | Upper gastrointestinal | 157,921 | G7 | −20% |
| stomach | series | | | |
| Injury of eye and orbit | Orbit [double | 157,752 | G7 | −20% |
| | exposure] (1 film) | | | |
| Female infertility | | 148,984 | G7 | −20% |
| Schizophrenia | | 146,901 | G7 | −20% |
| schizotypal and | | | | |
| delusional disorders | | | | |
| Epilepsy | | 145,844 | G7 | −20% |
| Other heart diseases | Chest [direct] (1 film) | 139,261 | G7 | −20% |
| Acquired deformities | | 134,995 | G7 | −20% |
| of limbs | | | | |
| Other maternal care | | 133,601 | G7 | −20% |
| related to fetus and | | | | |
| amniotic cavity and | | | | |
| possible delivery | | | | |
| problems | | | | |
| Heart failure | Chest [direct] (1 film) | 132,593 | G7 | −20% |
| Other vitamin | | 132,123 | G7 | −20% |
| deficiencies | | | | |
| Alcoholic liver disease | Abdomen (1 film) | 124,960 | G7 | −20% |
| Endometriosis | Female genitalia- | 122,658 | G7 | −20% |
| | hysterosalpingography | | | |
| Volume depletion | | 121,893 | G7 | −20% |
| Benign neoplasm of | Female genitalia- | 116,086 | G7 | −20% |
| ovary | hysterosalpingography | | | |
| Transient cerebral | Head (1 film) | 115,337 | G7 | −20% |
| ischaemic attacks and | | | | |
| related syndromes | | | | |
| Other infections and | | 114,776 | G7 | −20% |
| parasitic diseases | | | | |
| Single spontaneous | | 113,882 | G7 | −20% |
| delivery | | | | |
| Acute myocardial | Chest [direct] (1 film) | 110,328 | G7 | −20% |
| infarction | | | | |
| Diseases of appendix | Abdomen (1 film) | 110,244 | G7 | −20% |
| Parkinson's disease | | 105,834 | G7 | −20% |
| Complications | | 102,580 | G7 | −20% |
| predominantly related | | | | |
| to the puerperium and | | | | |
| other obstetric | | | | |
| conditions, not | | | | |
| elsewhere classified | | | | |
| Noninflammatory | Female genitalia- | 101,057 | G7 | −20% |
| disorders of ovary, | hysterosalpingography | | | |
| fallopian tube and | | | | |
| broad ligament | | | | |
| Cerebral palsy and | | 100,799 | G7 | −20% |
| other paralytic | | | | |
| syndromes | | | | |
| Acute pancreatitis and | Abdomen (1 film) | 100,660 | G7 | −20% |
| other diseases of the | | | | |
| pancreas | | | | |
| Other conditions | | 100,097 | G7 | −20% |
| originating in the | | | | |
| perinatal period | | | | |
| Hemorrhagic | | 98,978 | G7 | −20% |
| conditions and other | | | | |
| diseases of blood and | | | | |
| blood forming organs | | | | |
| Toxic effects of | | 98,159 | G7 | −20% |
| substances chiefly | | | | |
| nonmedicinal as to | | | | |
| source | | | | |
| Systemic connective | Neck, soft tissue (1 | 97,419 | G7 | −20% |
| tissue disorders | film) | | | |
| Intracerebral | Head (1 film) | 97,323 | G7 | −20% |
| hemorrhage | | | | |
| Other glomerular | Abdomen (1 film) | 97,302 | G7 | −20% |
| diseases | | | | |
| Atherosclerosis | | 96,677 | G7 | −20% |
| Crushing injuries and | Lower leg (1 film) | 94,003 | G7 | −20% |
| traumatic amputations | | | | |
| of specified and | | | | |
| multiple body regions | | | | |
| Malignant neoplasm of | Chest iordotic view | 91,295 | G7 | −20% |
| trachea, bronchus and | (1 film) | | | |
| lung | | | | |
| Paralytic ileus and | | 88,315 | G7 | −20% |
| intestinal obstruction | | | | |
| without hernia | | | | |
| Fracture of femur | Femur (1 film) | 86,602 | G7 | −20% |
| Malignant neoplasm of | Abdomen (1 film) | 86,472 | G7 | −20% |
| colon | | | | |
| Malignant neoplasm of | Plain radiograph of the | 86,098 | G7 | −20% |
| prostate | kidneys, ureters, and | | | |
| | bladder (1 film) | | | |
| Retinal detachments | | 84,546 | G7 | −20% |
| and breaks | | | | |
| Other congenital | Chest iordotic view (1 | 83,188 | G7 | −20% |
| malformations | film) | | | |
| Bronchiectasis | Chest iordotic view (1 | 79,522 | G7 | −20% |
| | film) | | | |
| Congenital | | 76,684 | G7 | −20% |
| malformations of the | | | | |
| circulatory system | | | | |
| Mental and | | 74,856 | G7 | −20% |
| behavioural disorders | | | | |
| due to use of alcohol | | | | |
| Other pregnancies | | 74,179 | G7 | −20% |
| with abortive outcome | | | | |
| Malignant neoplasm of | Abdomen (1 film) | 73,171 | G7 | −20% |
| liver and intrahepatic | | | | |
| bile ducts | | | | |
| (Crohn's disease and | Abdomen (1 film) | 65,911 | G7 | −20% |
| ulcerativecolitis | | | | |
| Malignant neoplasm of | Abdomen (1 film) | 64,277 | G7 | −20% |
| rectosigmoid junction, | | | | |
| rectum, anus and anal | | | | |
| canal | | | | |
| Other diseases of | | 56,380 | G7 | −20% |
| arteries, arterioles and | | | | |
| capillaries | | | | |
| Diverticular disease of | | 52,864 | G7 | −20% |
| intestine | | | | |
| Other congenital | Abdomen (1 film) | 52,170 | G7 | −20% |
| malformations of the | | | | |
| digestive system | | | | |
| Respiratory | Chest iordotic view | 51,956 | G7 | −20% |
| tuberculosis | (1 film) | | | |
| Alzheimer's disease | | 49,149 | G7 | −20% |
| Inguinal hernia | | 48,688 | G7 | −20% |
| Sexually trans mitted | | 47,874 | G7 | −20% |
| chlamydial diseases | | | | |
| Contraceptive | | 43,732 | G7 | −20% |
| management | | | | |
| Sepsis | | 43,048 | G7 | −20% |
| Other congenital | Carpal bone (1 film) | 39,593 | G7 | −20% |
| malformations and | | | | |
| deformations of the | | | | |
| musculoskeletal | | | | |
| system | | | | |
| Other malformations | Abdomen (1 film) | 38,779 | G7 | −20% |
| of the genitourinary | | | | |
| system | | | | |
| Carcinoma in situ of | Female genitalia- | 36,847 | G7 | −20% |
| cervix uteri | hysterosalpingography | | | |
| Other malignant | Plain radiograph of the | 36,762 | G7 | −20% |
| neoplasms of urinary | kidneys, ureters, and | | | |
| tract | bladder (1 film) | | | |
| Malignant neoplasm of | Plain radiograph of the | 34,547 | G7 | −20% |
| bladder | kidneys, ureters, and | | | |
| | bladder (1 film) | | | |
| Phlebitis, | | 32,022 | G7 | −20% |
| thrombophlebitis, | | | | |
| venous embolism and | | | | |
| thrombosis | | | | |
| Vitamin A deficiency | | 30,632 | G7 | −20% |
| Other malignant | Esophagography | 26,499 | G7 | −20% |
| neoplasm of digestive | | | | |
| organs | | | | |
| Malignant neoplasm of | Female genitalia- | 26,420 | G7 | −20% |
| cervix uteri | hysterosalpingography | | | |
| Other infections | | 26,246 | G7 | −20% |
| specific to the | | | | |
| perinatal period | | | | |
| Other malignant | Female genitalia- | 25,539 | G7 | −20% |
| neoplasms of female | hysterosalpingography | | | |
| genital organs | | | | |
| Non-Hodgkin's | | 25,128 | G7 | −20% |
| lymphoma | | | | |
| Inflammatory diseases | | 24,815 | G7 | −20% |
| of the central nervous | | | | |
| system | | | | |
| Female genital | | 24,635 | G7 | −20% |
| prolapse | | | | |
| Benign neoplasm of | Plain radiograph of the | 23,482 | G7 | −20% |
| urinary organs | kidneys, ureters, and | | | |
| | bladder (1 film) | | | |
| Chronic rheumatic | | 23,056 | G7 | −20% |
| heart disease | | | | |
| Mental retardation | | 22,762 | G7 | −20% |
| Stroke, not specified | Head (1 film) | 22,379 | G7 | −20% |
| as haemorrhage or | | | | |
| infarction | | | | |
| Salpingitis and | Female genitalia- | 21,780 | G7 | −20% |
| oophoritis | hysterosalpingography | | | |
| Leukaemia | | 20,927 | G7 | −20% |
| Malignant neoplasm of | Female genitalia- | 20,560 | G7 | −20% |
| other and unspecified | hysterosalpingography | | | |
| parts of uterus | | | | |
| Sequelae of injuries, | | 19,887 | G7 | −20% |
| of poisoning and of | | | | |
| other consequences | | | | |
| of externalcauses | | | | |
| Benign neoplasm of | | 19,525 | G7 | −20% |
| brain and other parts | | | | |
| of central nervous | | | | |
| system | | | | |
| Slow fetal | | 19,348 | G7 | −20% |
| growth, fetal | | | | |
| malnutrition and | | | | |
| disorders related to | | | | |
| short gestation and | | | | |
| low birth weight | | | | |
| Obstructed labour | | 19,317 | G7 | −20% |
| Malignant neoplasms | Conventional | 19,021 | G7 | −20% |
| of lip, oral cavity and | panoramic view | | | |
| pharynx | | | | |
| Malignant neoplasm of | Abdomen (1 film) | 18,947 | G7 | −20% |
| pancreas | | | | |
| lodine-deficiency- | | 18,642 | G7 | −20% |
| related thyroid | | | | |
| disorders | | | | |
| Other hernia | | 17,546 | G7 | −20% |
| Other respiratory | | 17,316 | G7 | −20% |
| disorders originating in | | | | |
| the perinatal period | | | | |
| Other malignant | | 16,654 | G7 | −20% |
| neoplasms of skin | | | | |
| Pulmonary embolism | Chest iordotic view | 16,132 | G7 | −20% |
| | (1 film) | | | |
| Hydrocele and | | 16,028 | G7 | −20% |
| spermatocele | | | | |
| Poisoning by drugs | | 15,729 | G7 | −20% |
| and biological | | | | |
| substances | | | | |
| Other malignant | | 15,128 | G7 | −20% |
| neoplasms of | | | | |
| lymphoid, | | | | |
| haematopoietic and | | | | |
| related tissue | | | | |
| Mumpus | | 15,119 | G7 | −20% |
| (Acute and rapidly | Plain radiograph of the | 14,458 | G7 | −20% |
| progressive nephritic | kidneys, ureters, and | | | |
| syndromes | bladder (1 film) | | | |
| Obesity | | 14,416 | G7 | −20% |
| Spontaneous abortion | | 13,795 | G7 | −20% |
| Other syphilis | | 13,790 | G7 | −20% |
| Congenital deformities | Foot (1 film) | 13,157 | G7 | −20% |
| of feet | | | | |
| Osteomyelitis | | 12,206 | G7 | −20% |
| Gonococcal infection | | 11,831 | G7 | −20% |
| Injury of other internal | Abdomen (1 film) | 11,130 | G7 | −20% |
| organs | | | | |
| Malignant neoplasm of | Head (1 film) | 10,970 | G7 | −20% |
| brain | | | | |
| Human | | 10,887 | G7 | −20% |
| immunodeficiency | | | | |
| virus [HIV] disease | | | | |
| Malignant neoplasm of | Neck, soft tissue (1 | 10,603 | G7 | −20% |
| mesothelial and soft | film) | | | |
| tissue | | | | |
| Oedema, proteinuria | | 10,540 | G7 | −20% |
| and hypertensive | | | | |
| disorders in | | | | |
| pregnancy, childbirth | | | | |
| and the puerperium | | | | |
| Malignant neoplasm of | Esophagography | 9,891 | G7 | −20% |
| oesophagus | | | | |
| Mental and | | 9,591 | G7 | −20% |
| behavioural disorders | | | | |
| due to other | | | | |
| psychoactive | | | | |
| substance use | | | | |
| Congenital deformities | Hip joint (1 film) | 9,364 | G7 | −20% |
| of hip | | | | |
| Other tuberculosis | Chest iordotic view (1 | 9,212 | G7 | −20% |
| | film) | | | |
| praevia, premature | | 9,112 | G7 | −20% |
| separation of placenta | | | | |
| and antepartum | | | | |
| haemorrhage | | | | |
| Arterial embolism and | | 8,678 | G7 | −20% |
| throm bosis | | | | |
| Redundant prepuce, | | 8,448 | G7 | −20% |
| phimosis and | | | | |
| paraphimosis | | | | |
| Other helminthiases | | 7,923 | G7 | −20% |
| Undescended testicle | | 7,867 | G7 | −20% |
| Malignant neoplasm of | Cervical spine (1 film) | 7,392 | G7 | −20% |
| larynx | | | | |
| Typhus fever | | 7,252 | G7 | −20% |
| Chromosomal | | 7,005 | G7 | −20% |
| abnormalities, not | | | | |
| elsewhere classified | | | | |
| Sequelae of | Chest iordotic view (1 | 6,888 | G7 | −20% |
| tuberculosis | film) | | | |
| Other malignant | Chest [direct] | 6,404 | G7 | −20% |
| neoplasms of | (1 film) | | | |
| respiratory and | | | | |
| intrathoracic organs | | | | |
| Other congenital | | 6,286 | G7 | −20% |
| malformations of the | | | | |
| nervous system | | | | |
| Blindness and low | | 6,144 | G7 | −20% |
| vision | | | | |
| Early syphilis | | 5,649 | G7 | −20% |
| Certain disorders | | 5,553 | G7 | −20% |
| involving the immune | | | | |
| mechanism | | | | |
| Malnutrition | | 5,404 | G7 | −20% |
| Cleft lip and cleft | | 5,155 | G7 | −20% |
| palate | | | | |
| Pneumoconiosis | Chest iordotic view (1 | 4,989 | G7 | −20% |
| | film) | | | |
| Amoebiasis | | 4,949 | G7 | −20% |
| Malignant neoplasm of | Neck, soft tissue (1 | 4,731 | G7 | −20% |
| bone and articular | film) | | | |
| cartilage | | | | |
| Postpartum | | 4,588 | G7 | −20% |
| hemorrhage | | | | |
| Fetus and newborn | | 4,077 | G7 | −20% |
| affected by maternal | | | | |
| factors and by | | | | |
| complications of | | | | |
| pregnancy, labour and | | | | |
| delivery | | | | |
| Other fluke infections | | 4,064 | G7 | −20% |
| Malignant melanoma | | 3,865 | G7 | −20% |
| of skin | | | | |
| Senility | | 3,028 | G7 | −20% |
| Birth trauma | | 2,828 | G7 | −20% |
| Acute hepatitis B | | 2,801 | G7 | −20% |
| Other malignant | | 2,662 | G7 | −20% |
| neoplasms of male | | | | |
| genital organs | | | | |
| multiple sclerosis | | 2,525 | G7 | −20% |
| Spina bifida | Entire spine (1 film) | 2,517 | G7 | −20% |
| Maltreatment | | 2,272 | G7 | −20% |
| syndromes | | | | |
| Congenital infectious | | 2,245 | G7 | −20% |
| and parasitic diseases | | | | |
| Hodgkin disease | | 2,043 | G7 | −20% |
| Chronic rheumatic | | 1,993 | G7 | −20% |
| heart disease | | | | |
| Malignant neoplasm of | | 1,971 | G7 | −20% |
| other parts of central | | | | |
| nervous system | | | | |
| Malignant neoplasm of | | 1,220 | G7 | −20% |
| eye and adnexa | | | | |
| Malaria | | 1,214 | G7 | −20% |
| Other arthropod-borne | | 1,025 | G7 | −20% |
| viral fevers and viral | | | | |
| haemorrhagic fevers | | | | |
| Whooping cough | | 947 | G7 | −20% |
| viral encephalitis | | 926 | G7 | −20% |
| Hookworm disease | | 894 | G7 | −20% |
| Intrauterine hypoxia | | 839 | G7 | −20% |
| and birth asphyxia | | | | |
| Medical abortion | | 668 | G7 | −20% |
| Sequelae of | | 494 | G7 | −20% |
| poliomyelitis | | | | |
| Other tetanus | | 394 | G7 | −20% |
| Fractures involving | | 317 | G7 | −20% |
| multiple body regions | | | | |
| Sequelae of | | 303 | G7 | −20% |
| malnutrition and other | | | | |
| nutritional deficiencies | | | | |
| Congenital absence, | | 299 | G7 | −20% |
| atresia and stenosis of | | | | |
| small intestine | | | | |
| Measles | | 290 | G7 | −20% |
| Typhoid and | | 262 | G7 | −20% |
| paratyphoid fevers | | | | |
| Rubella | | 242 | G7 | −20% |
| Shigellosis | | 206 | G7 | −20% |
| congenital syphilis | | 185 | G7 | −20% |
| Leprosy [Hansen's | | 133 | G7 | −20% |
| disease] | | | | |
| Asymptomatic human | | 108 | G7 | −20% |
| immunodeficiency | | | | |
| virus [HIV] infection | | | | |
| status | | | | |
| Haemolytic disease of | | 92 | G7 | −20% |
| fetus and newborn | | | | |
| Relapsing fevers | | 85 | G7 | −20% |
| Cholera | | 57 | G7 | −20% |
| Schistosomiasis | | 55 | G7 | −20% |
| Acute poliomyelitis | | 44 | G7 | −20% |
| Trachoma | | 41 | G7 | −20% |
| Meningococcal | | 40 | G7 | −20% |
| infection | | | | |
| Trypanosomiasis | | 27 | G7 | −20% |
| Dracunculiasis | | 18 | G7 | −20% |
| Rabies | | 17 | G7 | −20% |
| Echinococcosis | | 16 | G7 | −20% |
| Plague | | 10 | G7 | −20% |
| Sequelae of leprosy | | 10 | G7 | −20% |
| Onchocerciasis | | 9 | G7 | −20% |
| Brucellosis | | 8 | G7 | −20% |
| Yellow fever | | 3 | G7 | −20% |
| Leishmaniasis | | 2 | G7 | −20% |
| Tetanus neonatorum | | 2 | G7 | −20% |
| Filariasis | | 0 | G7 | −20% |
| Diphtheria | | 0 | G7 | −20% |

In the calculating of the value according to the market approach (S230), an effectiveness weight was calculated, and a unit cost was derived by adding 20% of a distribution for each disease in Table 6 to a cost derived through market approach. The valuation of the medical data (S200) may further include reflecting a distribution fee and reflecting other statutory taxes. Specifically, in the reflecting of the distribution fee, the distribution fee was set for structured data and unstructured data of the medical data. Specific standards thereof are shown in Table 7 below.

TABLE 7

| Types of data management | Weight |
|---|---|
| Structured data | 5% |
| Unstructured X-ray | 10% |
| Unstructured -voice | 15% |
| Unstructured-image | 20% |
| Unstructured-genom | 25% |

One or more embodiments are not limited thereto, but the reflecting of the other statutory taxes may include reflecting a value-added tax. The value of the medical data was valued by adding a value-added tax of 10%.

The performing of final valuation (S300) may include finally evaluating the value of the medical data calculated in the valuation of the medical data. The performing of final valuation may further include classifying medical data in which a disease is identifiable and medical data in which a disease is not identifiable.

Accordingly, in the final valuation, medical data in which a disease is not is identified may be valued as having 60% of value of medical data in which a disease is identifiable.

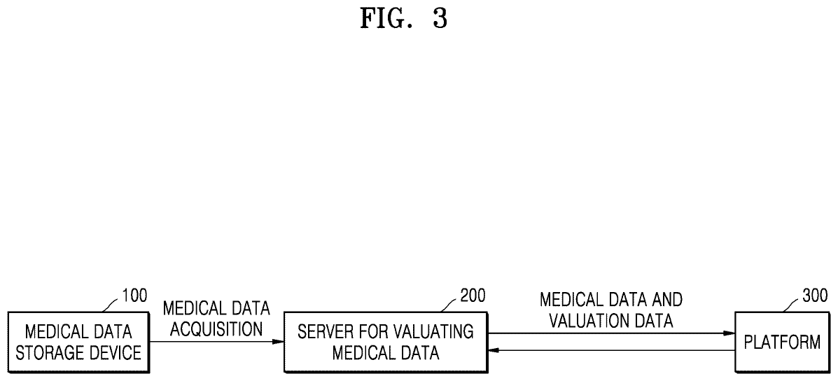

FIG. 3 is a block diagram of a system for evaluating the value of medical data, which includes a server for evaluating the value of medical data.

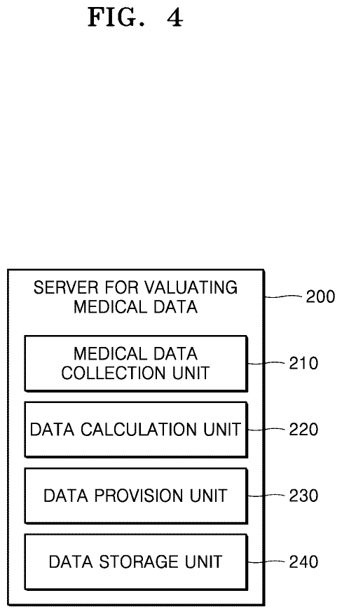

FIG. 4 is a block diagram of a server for evaluating the value of medical data.

Another aspect provides a server 200 for evaluating the value of medical data, which includes a medical data collection unit that acquires medical data, a data calculation unit 220 that determines value of the medical data by calculating the value of the acquired medical data according to a cost approach, market approach, and income approach, a data provision unit 230 that provides a calculated value evaluation result of the medical data to a platform or a mobile device, and a data storage unit 240 that stores the acquired medical data and the value evaluation result thereof.

The medical data collection unit may acquire various types of medical data for value evaluation. The received medical data is not limited thereto, but may be structured or unstructured data, preferably, unstructured X-ray data. Data acquired by the medical data collection unit may be valued by the data calculation unit.

The data calculation unit may value the medical data through a method of valuation according to an embodiment. Medical data value evaluation data derived by the data calculation unit may be transmitted or received by the data provision unit or stored by the data storage unit.

The data provision unit may transmit or receive the medical data value evaluation data to or from a platform 300.

The data storage unit may store the medical data or the medical data value evaluation data calculated by the data calculation unit.

The platform 300 is not limited thereto, but may be a cloud platform, a terminal, or a device, and as the terminal, a smartphone, a tablet computer, a laptop, or the like may be used. There are no restrictions on a platform through which value evaluation is to be identified or traded. The platform may be installed with an app for trading valuation data or may access a relevant internet page.

The server may include a communication interface, an input/output interface, a processor, and a memory. However, components of the server are not limited to the examples described above and may include more or fewer components than the components described above. In an embodiment, some or all of the communication interface, the input/output interface, the processor, and the memory may be implemented in the form of a single chip, and the processor may include one or more processors.

This research has been conducted with the support of the Korea Institute of Industrial Technology as “Development and Demonstration of Health Data Distribution Platform for Supporting Medical Device Companies Based on Data Curation” (Project No. 1415180073).

This research has been conducted with the support of the Ministry of Science and ICT as “AIM: AI-based Next-Generation Security Information Management Technique Application Cognitive Intelligence and Secure-Open Framework (S-OFW) Technology Development (Project No. 1711126334).

The reference numerals shown in the drawings of this specification may indicate the following components.
100: medical data storage device
200: server for valuating medical data
210: medical data collection unit
220: data calculation unit
230: data provision unit
240: data storage unit
300: platform

1. A method of valuation of medical data, the method comprising:
acquiring medical data;
valuation of the acquired medical data; and
performing a final valuation.

2. The method of claim 1, wherein the valuation of the medical data comprises:
a) calculating the value according to a cost approach;
d) calculating the value according to a market approach; and
d) calculating the value according to an income approach.

3. The method of claim 2, wherein the calculating the value according to a cost approach comprises calculating a scale of an institution in which the medical data is generated, and calculating a weight according to the calculated scale.

4. The method of claim 2, wherein the calculating the value according to the cost approach further comprises storing the medical data and calculating a management cost.

5. The method of claim 1, wherein the medical data is X-ray data.

6. The method of claim 1, wherein the performing of the final valuation further comprises classifying medical data in which a disease is identifiable and medical data in which a disease is not identifiable.

7. The method of claim 1, wherein, in the evaluation of the final value, the medical data in which a disease is not identified is valued as having 60% of value of medical data in which a disease is identifiable.

8. The method of claim 1, wherein the valuation of the medical data further comprises adding a weight to a distribution fee.

9. The method of claim 1, wherein the valuation of the medical data further comprises adding value-added tax according to a data transaction.

10. The method of claim 2, wherein the calculating the value according to the cost approach comprises calculating the value according to medical practice unit cost data.

11. The method of claim 2, wherein the valuation according to the market approach comprises reflecting a weight according to scarcity of a corresponding disease.

12. A server for valuation of medical data, the server comprising:
a medical data collection unit configured to acquire medical data;
a data calculation unit configured to calculate value of the medical data by calculating the value of the medical data acquired according to a cost approach, calculate a scale of an institution in which the medical data is generated, store the medical data, calculate a management cost, calculate the value according to a market approach, and calculate the value according to an income approach;
a data provision unit configured to provide a calculated value evaluation result of the medical data to a platform or a mobile device; and
a data storage unit configured to store the acquired medical data and the value evaluation result thereof.

13. The server of claim 12, wherein the medical data is X-ray data.
